(19) United States
(12) Patent Application Publication
(10) Pub. No.: US 2017/0328877 A1
(43) Pub. Date: Nov. 16, 2017

(54) Smart Power Outlet with Gas Detection Feature

(71) Applicant: Tushar Bajaj, Houston, TX (US)

(72) Inventor: Tushar Bajaj, Houston, TX (US)

(73) Assignee: Tushar Bajaj, Houston, TX (US)

(21) Appl. No.: 15/155,049

(22) Filed: May 15, 2016

(51) Int. Cl. G01N 33/00; G08B 17/10; G01M 3/04

(52) U.S. Cl. CPC G01N 33/0036; G01M 3/04; G08B 17/10

(57) ABSTRACT

One embodiment (FIGS. 1A-1E) of smart electrical power outlets combined with gas detection capability and of type that plugs into another electrical power outlet. The flow of electrical energy from a power inlet plug (114) to power outlets (102a, 102b) can be, depending on user configuration, turned on or turned off when a hazardous gas is detected by a gas sensor (150). The embodiment is a versatile device that can be installed in any location where a power outlet is available, and the device can be used to power other electrical devices such as ventilation fans or gas shut-off valve. In addition, a wireless communication module (140) is included so that an alert message can be sent to the user when a gas leak is detected. The embodiment also provides audio and visual alerts through an LED (110) and loudspeaker (136). Other embodiments are described and shown.

### BACKGROUND

### I. Field of the Invention

This invention relates to the field of gas monitoring, and more particularly to smart gas detection devices that provide additional functionality. The invention presents widgets that detect hazardous gases, communicate through a wireless medium and also act as smart power outlets.

### II. Description of the Related Art

Basic gas monitoring devices are well known in prior art. Generally, these devices can detect toxic or combustible gases. Such devices find applications in both residential and commercial spaces. Typically, these devices generate an alarm when the amount of a hazardous gas present in the environment goes above a pre-determined threshold.

Kidde's AC Powered, Plug-In CO/Gas Combination Alarm (Model: KN-COEG-3) is an example of the hazardous gas detection devices that are commonly available in market. However, if the consumer is not present in the vicinity of the device, he or she will never know that a gas leak related alarm has been triggered. Dungan, in U.S. Pat. No. 6,252,510 (2001), demonstrates an elaborate system that uses remote gas monitoring nodes. The monitoring nodes use wireless communication to transmit monitored gas levels and alert signals to a control center. Dungan's system is targeted towards large industrial applications; hence, it requires a lot of monetary investment and technical expertise for implementation. It is because of these drawbacks; such systems have not been readily used in either commercial or residential applications.

Other gas monitoring systems with additional functionality have also been proposed—for example, in U.S. Pat. No. 4,916,437 to Gazzaz (1990) and U.S. Pat. No. 8,511,345 to Bavishi (2013). Both the patents propose a gas monitoring system that detect airborne hydrocarbons and automatically shuts off a supply valve to stop the flow of gas from the gas source. Again, the applications of such systems are very limited and expensive because it would require the user to hire a professional to perform the installation and structural changes to the location where such systems are installed. Moreover, the remedial action taken by these systems, when combustible gas is detected, are limited to only shutting off the supply valve.

All the gas monitoring systems that are known in the prior art suffer from a number of disadvantages:

(a) Most of the gas monitoring systems that offer added functions and benefits are expensive and difficult to install.

(b) Consumers that use gas-monitoring systems that have audio-visual alarms only are at risk of not knowing when there is a gas leak in their household or workspace if they are not present in the vicinity of the monitoring system.

(c) Remedial action taken by the gas-monitoring systems at present is limited to shutting off the gas supply valve. Other remedial actions such as turning on a ventilation-fan or sending a message to fire department are also useful in the scenario of a gas leak.

### SUMMARY

The present invention is developed to address the problems faced by current state of the art gas detection devices. The embodiments of this invention comprise smart electrical power outlets, which can be configured by the user to either turn ON or turn OFF power output when a hazardous gas is detected. The embodiments of the invention are also versatile widgets that can be installed in any location where a standard power outlet is available. The embodiments also incorporate a wireless communication module so that the widget can send an alert message remotely to the user when a gas leak is detected.

There are many advantages of combining hazardous gas detection capability with one or more electrical power outlets in one widget. The power outlets can be used to power external devices that do not have hazardous gas detection capability themselves.

An advantage of wireless communication capability is that when a gas leak is detected the alert message wirelessly transmitted by the widget can be delivered anywhere to the consumer via the Internet. This functionality will allow the consumer to take proper precautions before entering the effected area. Furthermore, the wireless communication module can also be used to upload configuration parameters remotely to the device using a computer connected to another, compatible, wireless communication module.

Another advantage the present invention has over prior art is that it provides a compact, versatile and cost-effective device that can be easily installed and de-installed by a non-technical consumer.

It should be noted that the advantages listed here are for one or more aspects and not for the invention. Other benefits and advantages of the invention will become apparent to those skilled in the art upon a reading and understanding of the following detailed specification.

### BRIEF DESCRIPTION OF THE DRAWINGS

In the drawings, closely related figures have the same number but different alphabetic suffix. The provided drawings only depict certain embodiments of the invention and are not therefore to be considered to be limiting of the scope of the invention. The invention will now be described in greater detail by way of example with reference to the drawings, in which:

FIG. 1A is a perspective view of a first embodiment;

FIG. 1B is a front view of the embodiment shown in FIG. 1A;

FIG. 1C is a right-side view of the embodiment shown in FIG. 1A;

FIG. 1D is a rear view of the embodiment shown in FIG. 1A;

FIG. 1E is a left-side view of the embodiment shown in FIG. 1A;

FIG. 2A is a perspective view of a second embodiment;

FIG. 2B is a front view of the embodiment shown in FIG. 2A;

FIG. 2C is a right-side view of the embodiment shown in FIG. 2A;

FIG. 2D is a back view of the embodiment shown in FIG. 2A;

FIG. 2E is a left-side view of the embodiment shown in FIG. 2A;

FIG. 3 is a block diagram that illustrates key functional blocks of electronics used in the two embodiments shown in FIGS. 1A-1E and FIGS. 2A-2E;

FIG. 4 is a flow chart of gas detection portion of the process followed by the microcontroller or CPU used inside the two embodiments shown in FIGS. 1A-1E and FIGS. 2A-2E.

### DETAILED DESCRIPTION

I. Description

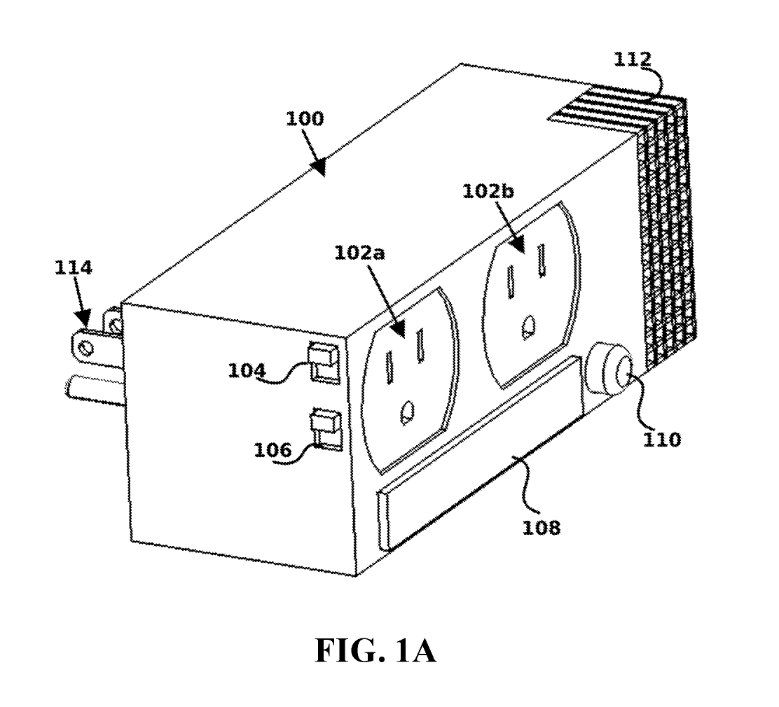

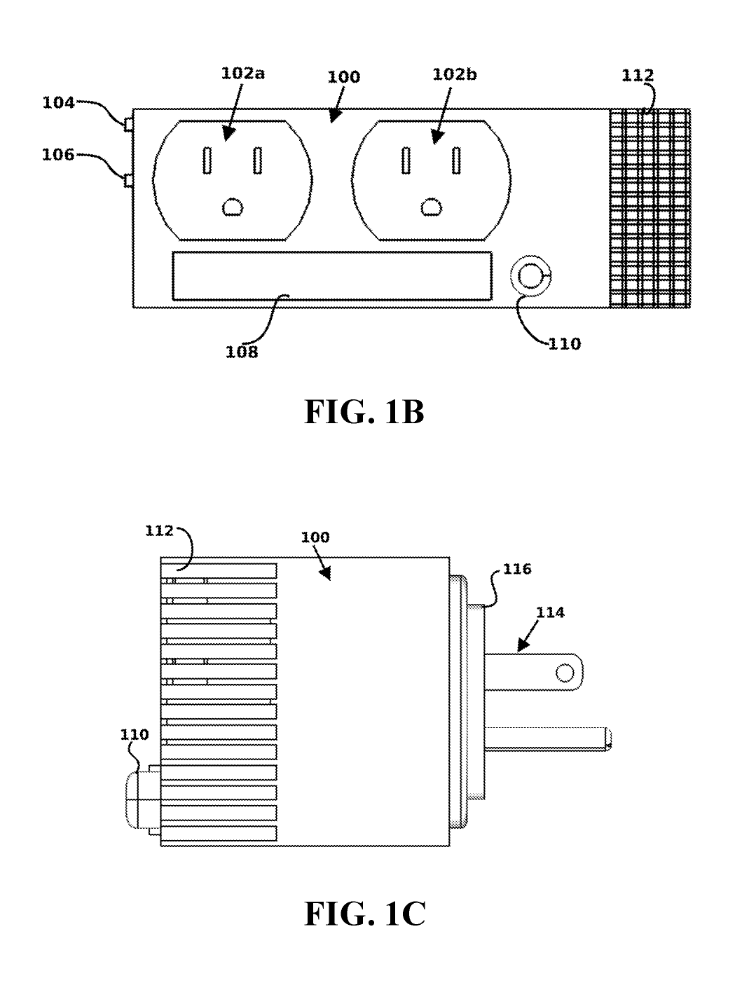

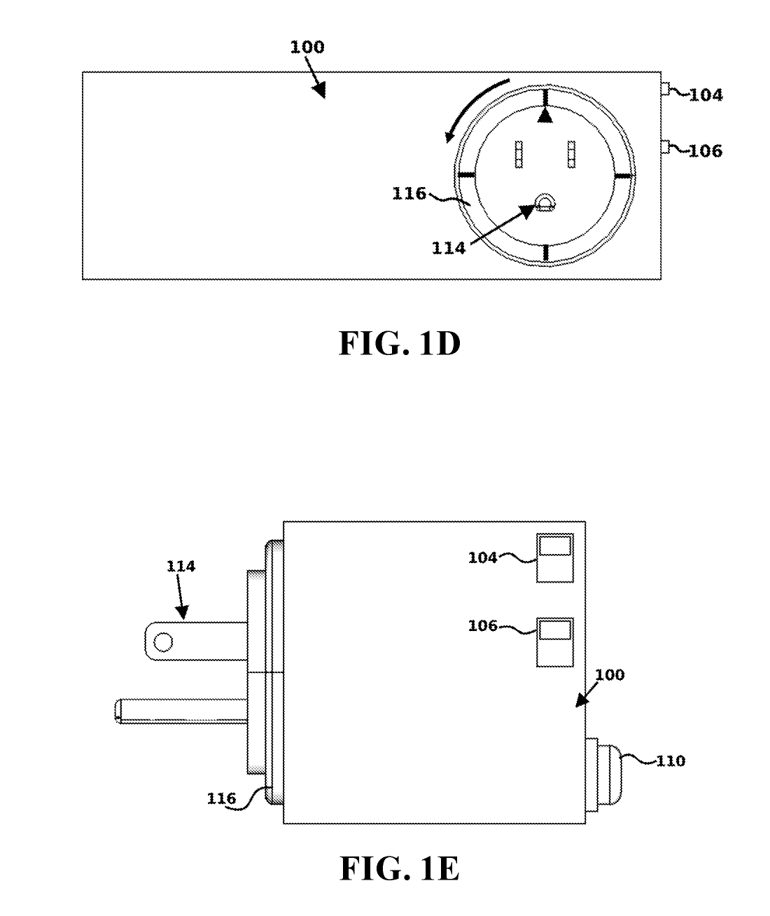

One embodiment of the invention is illustrated in the widget shown in FIG. 1A (perspective view), FIG. 1B (front view), FIG. 1C (right-side view), FIG. 1D (rear view) and FIG. 1E (left-side view). This widget demonstrates a plug-in version of Smart Power Outlet with Gas Detection Feature. The widget has a gas inlet window or opening 112 and a gas sensor or gas detector 150 is placed inside the inlet window. In the preferred embodiment the inlet window is mesh type; however, it can be of other types such as diagonal slits, circular slits etc. The purpose of this window is to allow sufficient airflow to the gas sensor, while preventing contaminants from entering the widget and inhibiting the sensor behavior.

I contemplate that the enclosure or housing 100 of the widget is made out of ABS plastic, but other rigid electrically non-conducting material such as UF, PVC, Acrylic etc. are also appropriate. I envision the overall dimension of the widget, not including the power inlet plug 114, is roughly 150 mm×50 mm×50 mm; and the enclosure can be 0.8 mm to 1.2 mm in wall thickness. However, the widget can be of different shapes and sizes.

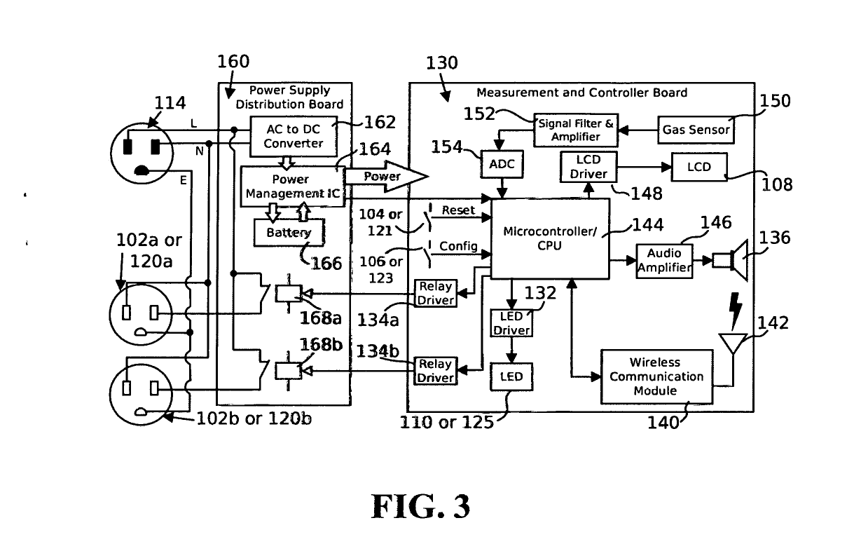

This embodiment includes two power outlet sockets 102a and 102b, which can be used to power any electrically powered device or appliance. One power inlet plug 114 is used to plug the widget into a power supply outlet. The power from the inlet plug is used to power all the electronics inside the widget as well as to power the two outlets 102a and 102b, through relays 168a and 168b. The rotating electrical contact disc 116 allows for the widget, when plugged into a power supply outlet, to be rotated to 0, 90, 180 or 270 degree positions. Due to the rotating feature, the gas inlet window 112 can always be pointed towards the possible source of gas leak. The enclosure 100 of the widget houses electronics and circuitry depicted in the functional block diagram given in FIG. 3. I contemplate that the electronics inside the widget can be divided between two sections: a Measurement and Controller Board 130 and a Power Supply Distribution board 160.

The Measurement and Controller Board 130 comprises of following parts:

Microcontroller or CPU 144 is used for processing all the data, and to perform control operations. Going forward the term microcontroller is used interchangeably with CPU.

Gas Sensor or Gas Detector 150 detects hazardous gas. The gas sensor used in this embodiment is Futurlec's MQ-5, which can detect natural gas. However, depending on the type of hazardous gas to be detected, the type of gas sensor used in the widget can be of other kind. The gas sensor can be of kind that detects Methane, Propane, Butane, Hydrogen, Carbon Monoxide, Chlorine, Sulfur Dioxide, Nitrogen Dioxide, or any other airborne contaminants or gas that can be deemed hazardous. Going forward the term gas sensor is used interchangeably with gas detector.

Signal Filter and Amplifier 152 conditions analog output of the gas sensor. This circuit will not be needed if the gas sensor used in the widget has a pre-conditioned output or a digital output.

Analog to Digital Converter (ADC) 154 converts the conditioned analog output of the gas sensor into a digital measurement. The digital measurement is then read by the microcontroller 144. ADC will not be needed if the gas sensor used in the widget already has a digital output, or if the microcontroller has an internal ADC.

Wireless Communication Module 140 and Antenna 142 transmit alerts and messages to a remote terminal or control center. Wireless communication can also be used for receiving commands and operational parameters. The wireless communication used in this embodiment is Wi-Fi based, however it can be based on other wireless communication protocols such as Z-Wave, Zigbee, GPRS, CDMA etc.

LCD 108 displays measurements, messages and prompts. The LCD driver 148 is used to drive the LCD from the microcontroller data output. Some LCDs may already have an integrated driver; in such cases a dedicated LCD driver is not needed.

LED 110 provides visual indication when a gas detection alarm has been generated. The LED driver 132 provides the current required to power the LED.

Loudspeaker 136 creates an audible alert when a gas detection alarm has been generated. The Audio Amplifier 146 is used to drive the loudspeaker.

Relay drivers 134a and 134b are used for driving relays 168a and 168b.

Toggling the Spring Switch 104 resets an alarm.

Single Pole Single Throw (SPST) switch 106 is used to switch the widget between configuration mode and operation mode.

The Power Supply Distribution board 160 comprises of following parts:

AC (Alternating Current) to DC (Direct Current) Converter 162 to converts the AC power supply to a usable DC power supply.

Rechargeable Battery 166 is a backup power source, used when there is a loss of AC power supply.

When AC power supply is available, the Power Management Integrated Circuit (PMIC) 164 directs the output from the AC to DC Converter 162 to power the electronics on the Measurement and Controller Board 130 and to recharge the battery 166. When there is a loss of AC power supply, the PMIC uses the backup battery to power the Measurement and Controller Board. The PMIC also indicates to the microcontroller 144 when there is a loss of AC power.

Relays 168a and 168b turn ON and OFF the flow of electricity from the input power supply to the power outlets 102a and 102b.

II. Operation

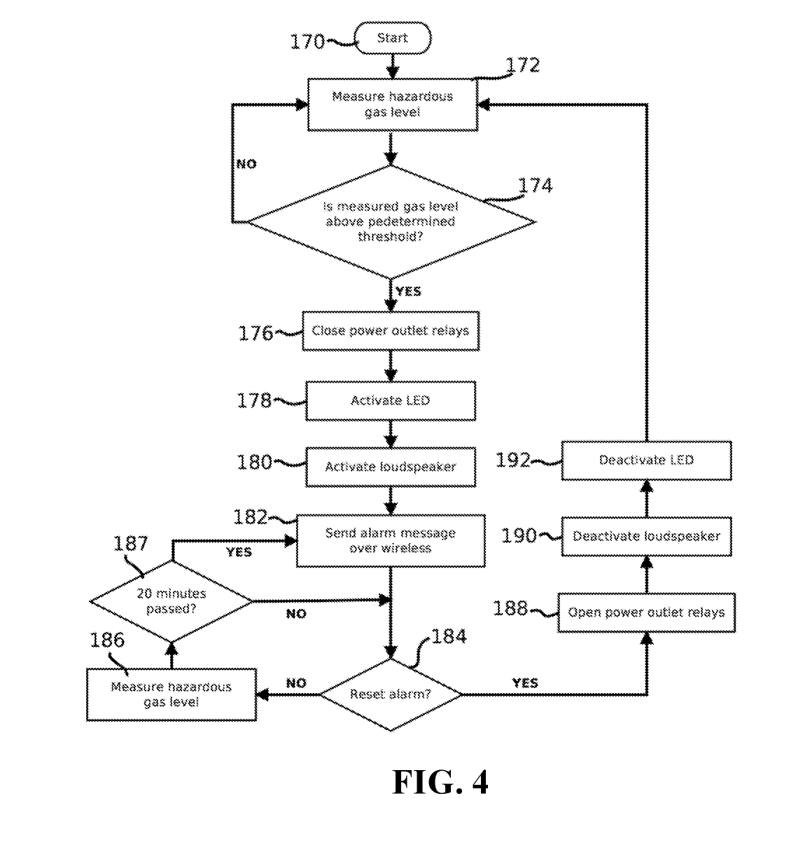

Flowchart in FIG. 4 describes the process followed by the microcontroller 144 in the embodiment to measure gas and make decisions based on the amount of gas measured. The measurement sequence starts at step 170. At step 172, microcontroller continuously reads gas measurement output of the ADC 154, and compares the amount of gas measured, typically in percentage by volume, against a predetermined threshold. When the amount of gas measured is above the threshold, the microcontroller closes the relays 168a and 168b. Closed relays switch ON the flow of electricity to the power outlets 102a and 102b respectively. After the relays are closed, the audio-visual alarms indications are generated through the loudspeaker 136 and LED 110. Following this, an alarm message is transmitted through the wireless communication module 140 to a remote terminal, wireless router or a data relay center. At step 184, the microcontroller checks if the alarm has been reset, either through the Spring Switch 104 or through a reset command received via wireless communication module. If the alarm has been reset, the microcontroller proceeds to step 188 and opens the relays 168a and 168b to turn OFF the flow of electricity to the power outlets 102a and 102b respectively. After relays are opened, the loudspeaker 136 and LED 110 alarm indications are deactivated; then the microcontroller goes back to step 172. However, if the alarm has not been reset, the microcontroller continues to step 186 to read the gas measurement output from the ADC 154. In this state, the microcontroller also sends an alarm message through the wireless communication module 140 at every 20 minutes intervals.

When the SPST switch 106 is in open position, the widget performs normal operation as mentioned in the FIG. 4 flowchart. However, when the switch is closed, the widget enters configuration mode. In configuration mode, the operator or user can upload operating parameters into the widget through the wireless communication module. I contemplate that the operating parameters could include: information for connecting to a Wi-Fi network, selection of alarm message transmission method and relevant configuration, time interval at which transmission of the alarm message is repeated, and default state of the relays during alarm and no-alarm conditions. User can even configure the relays to be always closed, in which case the power outlets of the widget will just act as normal power outlets, thereby providing additional power outlets for use.

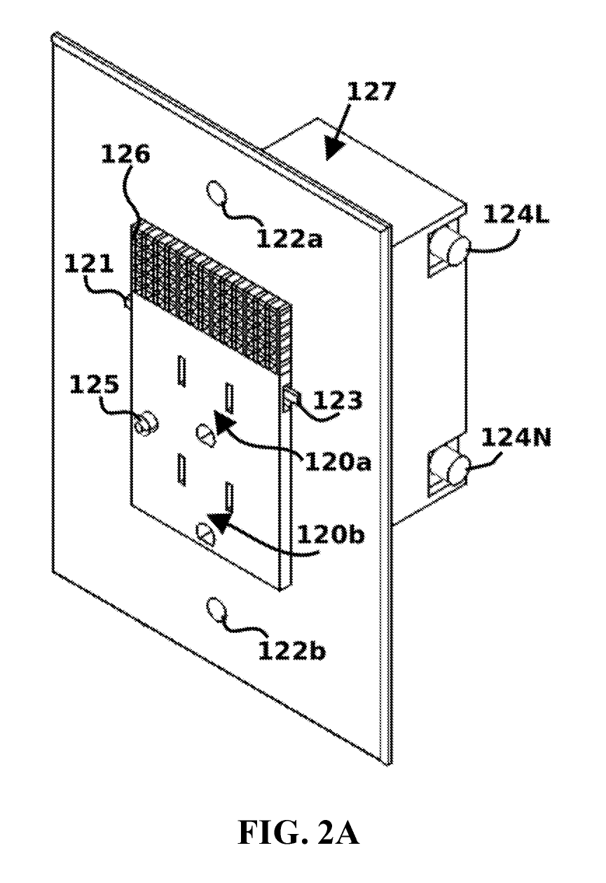

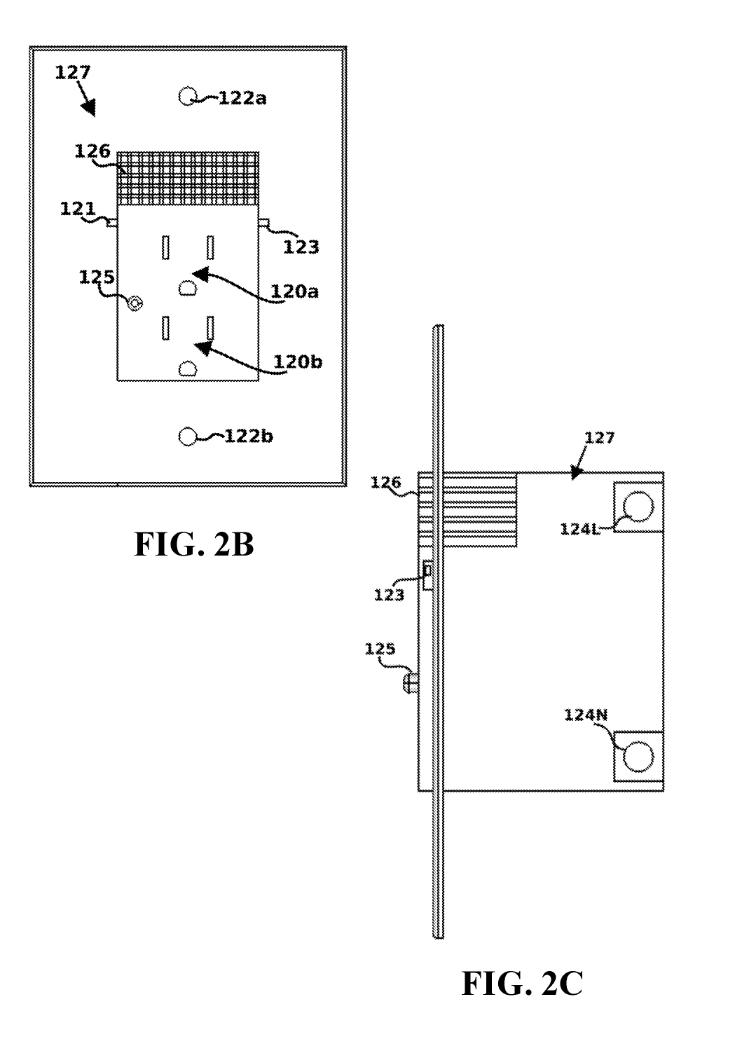

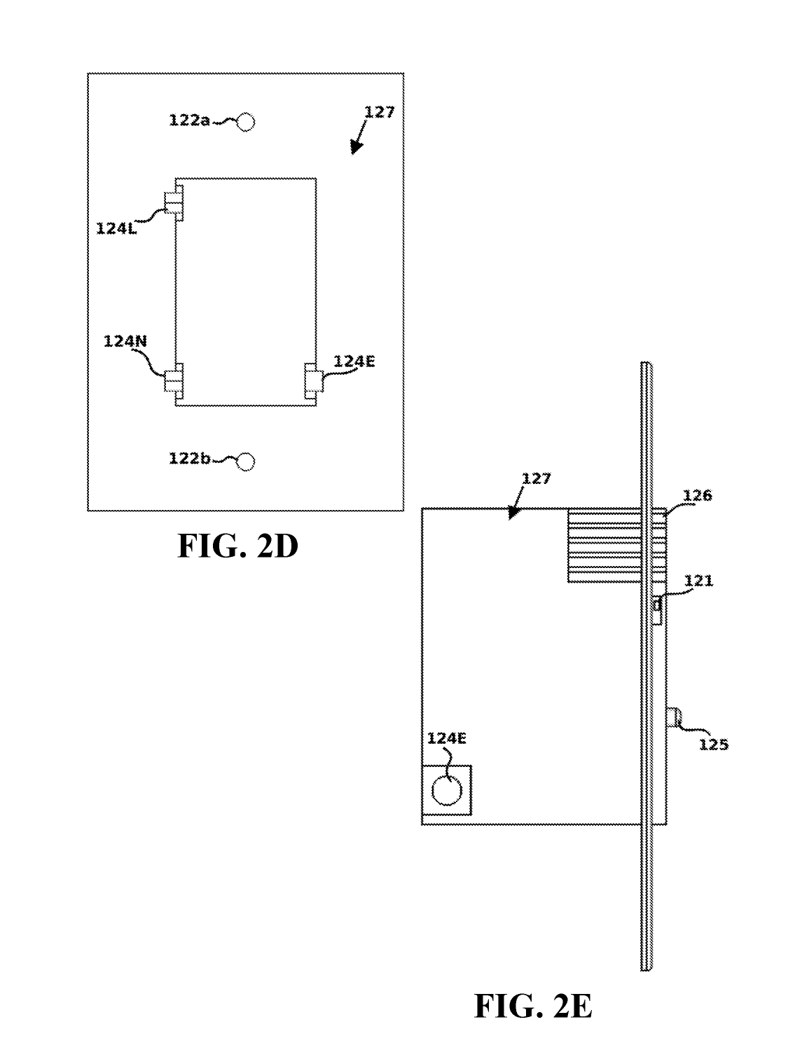

Second embodiment of the invention is illustrated in the widget shown in FIG. 2A (perspective view), FIG. 2B (front view), FIG. 2C (right-side view), FIG. 2D (rear view) and FIG. 2E (left-side view). This widget demonstrates a version of Smart Power Outlet with Gas Detection Feature that can be installed inside a wall cavity or a standard wall power outlet box. The mounting holes 122a and 122b are used for installing the widget inside the wall power outlet box. This embodiment also has a gas inlet window 126, two power outlet sockets 120a and 120b, one LED 125, one spring switch 121 and one SPST switch 123. I contemplate that the enclosure 127 of this embodiment is also made of the materials similar to the first embodiment shown in FIGS. 1A-1E. Since this embodiment is installed inside a wall power outlet box, the AC power supply wires of Line, Neutral and Earth can be connected to 124L, 124N and 124E terminals respectively. The enclosure 127 of this embodiment houses same electronics and circuitry as used in the first embodiment, except that instead of a power inlet plug 114, this embodiment uses terminals 124L, 124N and 124E. Overall functioning of this embodiment is same as the first embodiment.

### Ramifications And Scope

From the description above, a number of advantages of Smart Power Outlet with Gas Detection Feature become evident. The smart power outlets can be used to power external devices, which do not have gas detection capability. Such devices can be a gas shut-off valve, a ventilation fan or a fire-department alerting device.

By configuring the first embodiment to keep the power outlet relays always closed, the widget can also function as an extension that provides additional power outlets. The embodiments presented are versatile, they can be installed anywhere in residential and commercial space as long as a power source is available. They do not require expensive and elaborate installation. The widgets can be used and implemented by anyone with non-technical background.

Due to wireless communication capability the embodiments can send an alert message to the user no matter where the user is via email, SMS or any other internet-connected communication medium. The embodiments can also be configured to communicate with a remote control center or Internet-of-Things devices. The wireless message can also be used to remotely turn off a gas supply valve also connected to a compatible wireless communication module, without requiring installation of additional wiring or equipment.

While my above description of the embodiments contains much specificity, these should not be construed as limitation on the scope, but rather as an exemplification of several embodiments thereof. Many other variations are possible. For example:

(a) Electrical power outlets and inlets used in the embodiments are Type B (as per International Electrotechnical Commission standard), however, they can be of different types such as Type A, C, D or E.

(b) The number of electrical power outlets can be different.

(c) The number and type of gas sensors used in the embodiments can vary. The type of gas sensor used depends on the type of hazardous gas that is being detected; typically the gas sensors are catalytic, electrochemical or infrared based.

(d) The embodiments may not include a rechargeable battery.

(e) The wireless communication protocol used by the embodiments can vary.

Accordingly, the scope should be determined not by the embodiments illustrated, but by the appended claims and their legal equivalent.

I claim:

1. A gas detection widget, comprising:
a. means for connecting to a source of electrical energy,
b. means for transferring electrical energy from the source to a electrically powered device,
c. means for detecting one or more hazardous gas particles.

2. The gas detection widget of claim 1, further comprising means for controlling the transfer of electrical energy the source to said electrically powered device.

3. The gas detection widget of claim 1, further comprising communication means for transmitting and receiving data from one or more external computer.

4. The gas detection widget of claim 3, wherein said communication means comprises wireless communication.

5. The gas detection widget of claim 1, wherein means for detecting hazardous gases is selected from the group consisting of combustible and toxic gas sensors.

6. The gas detection widget of claim 1, further comprising an opening to allow airflow to the gas sensor.

7. The gas detection widget of claim 1, further comprising power supplying means for powering said gas detection widget from said source of electrical energy.

8. The gas detection widget of claim 1, further comprising means for storing electrical energy, whereby the storage means is used to power said gas detection widget during loss of said source of electrical energy.

9. The gas detection widget of claim 1, further comprising computer processor means for processing data.

10. The gas detection widget of claim 1, further comprising means for producing audible indication of an alarm.

11. The gas detection widget of claim 1, further comprising means for producing visual indication of an alarm.

12. The gas detection widget of claim 1, further comprising means for plugging into an electrical power outlet.

13. The device of claim 12, further comprising rotation means for rotating said gas detection widget about an axis perpendicular to the front face of said electrical power outlet.

14. The gas detection widget of claim 1, wherein said gas detection widget is placed inside a wall cavity.
